(19) United States
(12) Patent Application Publication
(10) Pub. No.: US 2024/0058115 A1
(43) Pub. Date: Feb. 22, 2024

(54) SUPPORT STRUCTURE FOR ARTIFICIAL EYE LENS

(71) Applicants: Dae Yune KIM, Busan (KR); Soo Eun KIM, Busan (KR)

(72) Inventors: Dae Yune KIM, Busan (KR); Soo Eun KIM, Busan (KR); Eun Joo LEE, Busan (KR)

(21) Appl. No.: 18/260,780

(22) PCT Filed: Jan. 13, 2021
(86) PCT No.: PCT/KR2021/000445

§ 371 Date: Jul. 7, 2023

(51) Int. Cl. A61F 2/16

(52) U.S. Cl. CPC A61F 2/16; A61F 2002/1686; A61F 2002/16902

(57) ABSTRACT

The present disclosure relates to a support structure including a frame extending in an arc shape concentric with a lens of the artificial eye lens, having an opening portion therein, and disposed adjacent to a rim of the lens of the artificial eye lens or disposed to support a rim of one side surface of the lens, a plurality of support parts disposed to be spaced apart from one another in a circumferential direction of the frame and configured to support the artificial eye lens to prevent the artificial eye lens from separating from the support structure, and a plurality of fixing parts extending outward from the frame in a diameter direction of the frame and having tips inserted and fixed into a tunnel formed in a sclera of the eyeball.

### CROSS-REFERENCE TO RELATED APPLICATION

This application is a National Phase Entry Application of pending PCT International Application No. PCT/KR2021/000445, filed on Jan. 13, 2021, the entire contents of which are hereby incorporated by references in its entirety.

### TECHNICAL FIELD

The present invention relates to a support structure for an artificial eye lens, and more particularly, to a support structure for supporting an artificial eye lens in an eyeball without inserting and seating the artificial eye lens into a capsular sac.

### BACKGROUND ART

As an ophthalmological method of treating an abnormal eye lens with a disease such as a cataract, a surgical procedure is mainly performed to remove contents in the eye lens positioned inside a capsular sac and insert an artificially manufactured intraocular lens (artificial eye lens) into a space in the capsular sac.

There has been mainly used an artificial eye lens including a lens substituted for a person's eye lens, and a haptic part configured to support the lens in the capsular sac. A type of artificial eye lens, which is mainly used, is illustrated in FIG. 1.

A haptic part 120 is elongated from a rim of a lens 110 and configured to support the lens while adjoining an inner rim of the capsular sac at a tip side, and two or four haptic parts formed in opposite directions have been mainly used. FIG. 1 illustrates the two haptic parts formed to be linearly symmetric.

A surgical procedure of inserting the artificial eye lens is performed by circularly incising a part of a front portion of the capsular sac, separating the capsular sac from eye lens contents, cutting, suctioning, and removing the eye lens contents, and then inserting the artificial eye lens. During the surgical procedure, damage to the capsular sac, congenital factors, or other diseases sometimes cause a state in which the capsular sac cannot accommodate and support the artificial eye lens.

In this case, there is used a method of fixing the artificial eye lens to an eyeball by connecting a thread to the lens rim or the haptic part of the artificial eye lens, extending the thread to the outside of a sclera, and fixing the thread to the sclera.

According to this surgical method, the artificial eye lens is supported in the eyeball by the thread, such that the artificial eye lens is not positioned in a position or disposed in a warped state. For this reason, there often occurs a situation in which the artificial eye lens cannot exhibit its own function, and sufficient eyesight cannot be ensured.

In particular, because the thread for fixing the artificial eye lens is sutured and fixed to the sclera, bacteria enter the eyeball from the outside through the thread, which causes a disease.

To solve the above-mentioned problems, there was proposed the invention disclosed in Korean Patent No. 10-1555298 (Document 1) entitled ‘Artificial Capsular Sac’.

The invention disclosed in Document 1 discloses a structure in which a circular thin film is formed to be substituted for a capsular sac, and the thin film is fixed to a ciliary body in an eyeball by knotting a fixing thread.

However, according to the structure, the process of disposing the artificial eye lens in the thin film is very difficult and requires a large amount of time. As a result, because the thin film is fixed to the eyeball by the thread, there also occurs a problem in that the thin film cannot be disposed at a position of the eye lens in the eyeball, and the thin film is warped or separated from a proper position. In particular, the process of fixing the thin film to the ciliary body by knotting the thread is very difficult and requires a significantly large amount of time.

### DISCLOSURE

### Technical Problem

The present invention has been made in an effort to solve the above-mentioned problems in the related art, and an object of the present invention is to provide a support structure configured to support an artificial eye lens in an eyeball.

Specifically, the object of the present invention is to provide a support structure used to support the artificial eye lens by fixing the artificial eye lens to an eyeball, i.e., a support structure that supports the artificial eye lens in the eyeball so that the artificial eye lens is not warped, or a lens of the artificial eye lens is not separated from a proper position without inserting an artificial eye lens into a capsular sac.

In particular, another object of the present invention is to provide a support structure that enables a surgical procedure of fixing an artificial eye lens to an eyeball to be simply performed, and enables a surgical procedure of inserting the artificial eye lens to be easily performed without concern of infection after the surgical procedure.

### Technical Solution

The above-mentioned technical problems of the present invention are solved by a support structure according to one aspect of the present invention that supports an artificial eye lens in an eyeball, the artificial eye lens being implanted into the eyeball so as to be substituted for an eye lens.

The support structure according to one aspect of the present invention includes: a frame extending in an arc shape concentric with a lens of the artificial eye lens, having an opening portion therein, disposed adjacent to a rim of the lens of the artificial eye lens or disposed to support a rim of one side surface of the lens; a plurality of support parts disposed to be spaced apart from one another in a circumferential direction of the frame and configured to support the artificial eye lens to prevent the artificial eye lens from separating from the support structure; and a plurality of fixing parts extending outward from the frame in a diameter direction of the frame and having tips inserted and fixed into tunnels formed in a sclera of the eyeball, in which the artificial eye lens includes the lens, and a plurality of haptic parts extending outward from the lens in a diameter direction of the lens and configured to come into contact with a capsular sac, and in which the artificial eye lens is disposed on the frame so that the haptic parts each protrude between the support parts.

The support structure configured as described above is inserted into the eyeball through an incised portion of a cornea that is made by incising the cornea. The frame of the support structure may be folded and inserted when the frame is inserted. The support structure is provided rearward of an iris so that the frame is concentric with a pupil.

Tunnels, into which the tips of the fixing parts of the support structure are inserted and fixed, are formed in a sclera. The tips of the fixing parts are inserted into the tunnels of the sclera through the sclera and then sutured. Therefore, the support structure of the present invention is fixed in the eyeball.

The artificial eye lens is inserted through the incised portion of the cornea and fixed to the support structure so that the lens of the artificial eye lens is supported on the support parts. The haptic part of the artificial eye lens is disposed to protrude between the support parts. Therefore, the artificial eye lens is supported by the support structure of the present invention and implanted into the eyeball.

According to the configuration of the support structure of the present invention, the artificial eye lens is implanted and fixed into the eyeball without being supported by the capsular sac.

In addition, the support structure of the present invention is fixed to the eyeball without means for fixing the support structure to the eyeball. In particular, because the fixing part of the support structure is inserted and fixed into the tunnel formed in the sclera, it is not necessary to perform a process of fixing the support structure by allowing a suture thread to penetrate the sclera during an implantation procedure. Therefore, the surgical procedure is simplified, and a risk of infection after the surgical procedure is less.

In addition, even in case that the capsular sac is damaged during the surgical procedure of inserting the artificial eye lens into the capsular sac, a procedure of supporting the already inserted artificial eye lens in the eyeball may be performed by inserting the support structure of the present invention into the eyeball.

There may be four embodiments of the support structure of the present invention.

In a first embodiment of the support structure of the present invention, the support parts may each include: a first portion extending from the frame and being in contact with one side surface of the artificial eye lens that faces one surface of the frame; and a second portion extending from a tip of the first portion and being in contact with a side surface of the lens opposite to one side surface of the lens, and the artificial eye lens may be supported as rims of two opposite surfaces of the lens of the artificial eye lens are in contact with the first and second portions of the support part.

In a second embodiment of the support structure of the present invention, the support parts may each include: a first portion extending from the frame and being in contact with one side surface of the lens of the artificial eye lens that faces one surface of the frame; and a second portion extending from the frame and being in contact with a side surface of the lens opposite to one side surface of the lens, and the artificial eye lens may be supported as rims of two opposite surfaces of the lens of the artificial eye lens are in contact with the first and second portions of the support part.

In a third embodiment of the support structure of the present invention, a circumference of one side surface of the artificial eye lens may be in contact with one surface of the frame, and each of the support parts may extend from the frame and be configured to come into contact with a side surface of the artificial eye lens opposite to one side surface of the artificial eye lens, such that the artificial eye lens may be supported as rims of two opposite surfaces of the lens are in contact with one surface of the frame and the support parts.

According to the embodiments of the support structure of the present invention, the rim of the lens of the artificial eye lens is fixed and supported by the frame and the support part or by only the support part of the support structure of the present invention. Because the support part may be just an integrated part protruding and extending from the frame without being configured be separated from the frame, the frame and the support part may be integrated by simple injection molding.

In addition, the process of supporting and fixing the artificial eye lens onto the support structure is performed in the eyeball. In the above-mentioned embodiments of the present invention, because the support parts are spaced apart from one another in the circumferential direction of the frame and protrude and extend, the artificial eye lens may be supported by the support structure by a simple process of inserting the rim of the lens of the artificial eye lens between the frame and the support part or into the support part.

A surgical procedure in the related art, which has been performed to date to fix an artificial eye lens, i.e., a surgical procedure of inserting an artificial eye lens with an attached haptic part into a capsular sac so that the haptic part is spread and supported by the capsular sac or a surgical procedure of fixing a thin film, which is substituted for a capsular sac, to an eyeball by means of a thread and inserting and fixing an artificial eye lens into the thin film is very difficult and time consuming.

In contrast, according to the configuration of the support structure of the present invention, the support structure is fixed to the eyeball just by performing the simple process of inserting the support structure into the eyeball and then inserting the fixing part into the tunnel formed in the sclera. Further, the artificial eye lens is supported by and fixed to the support structure by performing the simple process of fitting the rim of the lens of the artificial eye lens with the support part of the support structure inserted into the eyeball.

Therefore, according to the present invention, the process of inserting and fixing the support structure into the eyeball and the process of supporting the artificial eye lens on the fixed support structure may be performed very simply and quickly. In addition, in the present invention, almost any type of artificial eye lens may be supported, such that the support structure of the present invention may be implanted into the eyeball and support the inserted artificial eye lens without removing the artificial eye lens that was inserted and dislocated previously.

The support structure according to another aspect of the present invention includes: a frame extending in an arc shape concentric with a lens of the artificial eye lens, having an opening portion therein, disposed adjacent to a rim of the lens of the artificial eye lens or disposed to support a rim of one side surface of the lens; a plurality of support parts disposed to be spaced apart from one another in a circumferential direction of the frame and configured to support the artificial eye lens to prevent the artificial eye lens from separating from the support structure; and a plurality of fixing parts extending outward from the frame in a diameter direction of the frame and having tips inserted and fixed into tunnels formed in a sclera of the eyeball, in which the artificial eye lens includes the lens, and a plurality of haptic parts extending outward from the lens in a diameter direction of the lens and configured to come into contact with a capsular sac, in which the support parts are each formed in a ring shape fixed to the frame, and in which the artificial eye lens is supported as the haptic parts of the artificial eye lens are inserted into the rings.

In this configuration, the haptic part, which constitutes the artificial eye lens, is inserted and fixed into the ring of the support structure, such that the artificial eye lens may be more securely supported and more assuredly prevented from separating.

Meanwhile, the entire support structure of the present invention may be formed by injection molding using synthetic resin, and the fixing part may have higher rigidity than the frame.

In case that the frame has lower rigidity than the fixing part, the process of inserting the frame is easily performed because the frame may be folded and inserted during the process of incising the cornea and inserting the support structure of the present invention through the incised portion of the cornea. Further, because the fixing part has higher rigidity than the frame, the stability of the state in which the frame and the support part are supported by the sclera is improved.

According to the additional feature of the present invention, in the support structure of the present invention, a tip side of the fixing part has the same curvature as the sclera and is formed along a curved line concentric with the frame.

According to the above-mentioned configuration, the tip of the fixing part inserted into the tunnel formed in the sclera is matched with the shape of the sclera, such that the tip of the fixing part may be easily inserted into the tunnel of the sclera and seated properly after being inserted.

However, in the support structure of the present invention, the fixing part is made of a material having elasticity, such that the fixing part may be inserted and seated into the tunnel of the sclera even though the tip side of the fixing part is different in curvature from the sclera or formed straight, or the tip side of the fixing part is not concentric with the frame.

According to the additional feature of the present invention, in the support structure of the present invention, a thread and a needle may be sequentially coupled to the tip of the fixing part.

During the surgical procedure of implanting the support structure according to the present invention into the eyeball, the fixing part needs to pass through the sclera through the hole formed in the sclera, and the tip needs to be inserted into the tunnel formed in the sclera from the outside.

According to the additional feature of the present invention, because the thread and the needle are coupled to the tip of the fixing part, the thread and the needle coupled to the tip of the fixing part are inserted into the eyeball first, and then the needle protrudes from the outer surface of the sclera through the hole formed in the sclera at the time of inserting the support structure of the present invention into the eyeball. Next, when the needle protruding from the outer surface of the sclera is pulled after the remaining elements of the support structure are inserted into the eyeball, the thread is pulled, and the tip of the fixing part coupled to the thread protrudes from the outer surface of the sclera through the hole of the sclera. The needle may be used to insert the tip of the fixing part, which protrudes as described above, into the tunnel formed in the sclera. The thread and the needle are removed from the tip of the fixing part after being used.

As described above, according to the additional feature of the present invention, the surgical procedure of fixing the support structure according to the present invention to the sclera may be easily performed.

### DESCRIPTION OF DRAWINGS

FIG. 1 is a view illustrating an artificial eye lens that is typically used.

FIG. 2 is a top plan view illustrating a state in which an artificial eye lens is supported by a support structure according to a first embodiment of the present invention.

FIG. 3 is a longitudinal sectional view taken along line A-A in FIG. 2.

FIG. 4 is a transverse cross-sectional view of an eyeball that illustrates a state in which a support structure for an artificial eye lens according to the first embodiment of the present invention is inserted into and fixed to an eyeball.

FIG. 5 is a longitudinal sectional view taken along line A-A in FIG. 2 and illustrating a first modified example of the support structure according to the first embodiment of the present invention.

FIG. 6 is a longitudinal sectional view taken along line A-A in FIG. 2 and illustrating a second modified example of the support structure according to the first embodiment of the present invention.

FIG. 7 is a top plan view illustrating a state in which an artificial eye lens is supported by a support structure for an artificial eye lens according to a second embodiment of the present invention.

FIG. 8 is a longitudinal sectional view taken along line B-B in FIG. 6.

FIG. 9 is a view of the support structure when viewed along arrow C in FIG. 6.

### BEST MODE

Hereinafter, as specific details for carrying out the present invention, a configuration and operation of a support structure according to embodiments of the present invention will be specifically described with reference to the drawings.

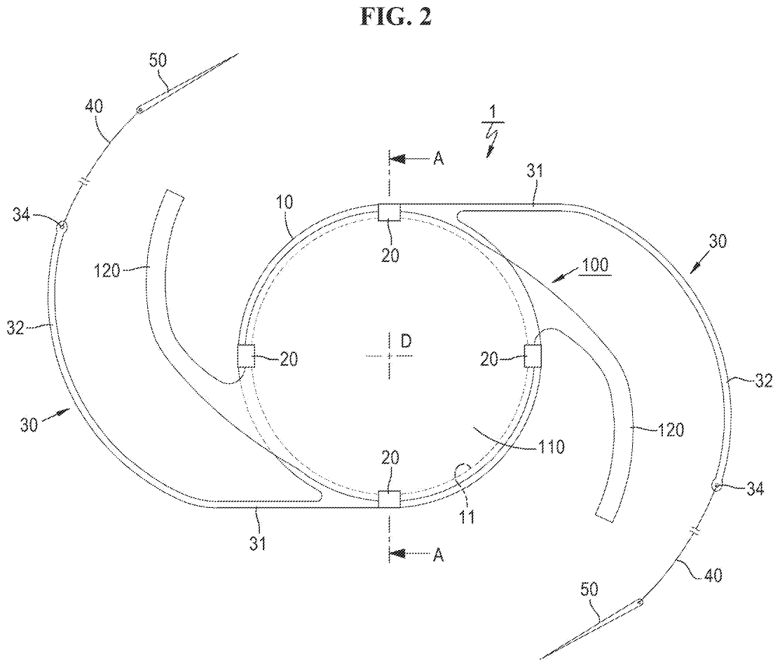

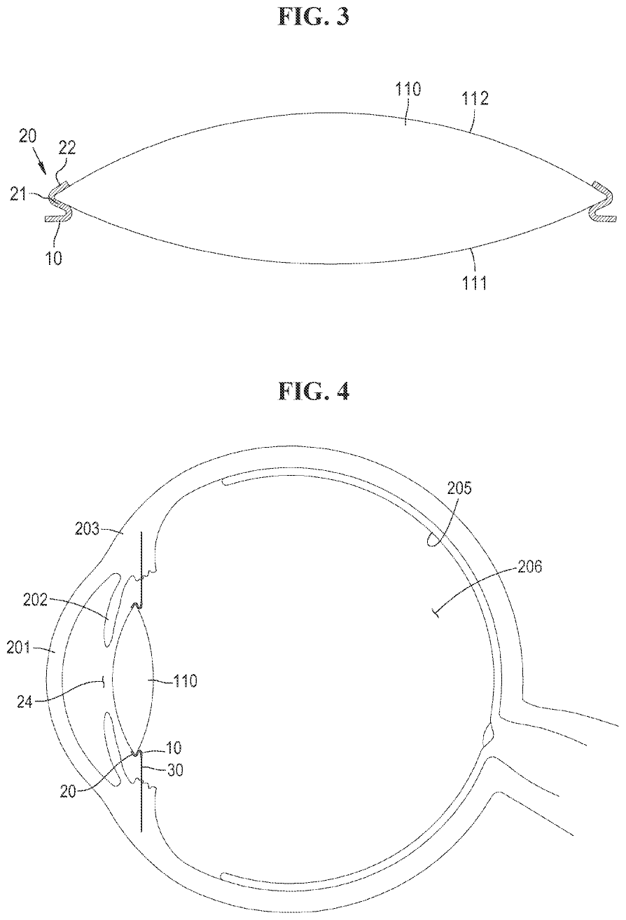

First, a configuration of a support structure 1 of a first embodiment will be described with reference to FIGS. 2 and 3.

The support structure 1 of the first embodiment includes: a frame 10 extending in an arc shape concentric with a lens 110 of an artificial eye lens 100 and having therein an opening portion 11 having a diameter larger than a maximum diameter of a pupil, the frame 10 being disposed to face one side surface of the artificial eye lens; four support parts 20 disposed to be spaced apart from one another in a circumferential direction of the frame 10 and configured to support the artificial eye lens to prevent the artificial eye lens from separating from the support structure 1; a plurality of fixing parts 30 extending outward from the frame 10 in a diameter direction of the frame 10 and having tips inserted and fixed into tunnels formed in a sclera of an eyeball; and threads 40 each having one end fixed to the tip of each of the fixing parts 30, the threads 40 extending from the tips of the fixing parts 30; and needles 50 coupled to tips of the threads 40.

In the first embodiment, the frame 10, the support part 20, and the fixing part 30 are integrated by injection molding. PMMA (polymethyl methacrylate), polypropylene, polyimide, silicone, acrylic resin, and the like may be used as a material of the frame 10, the support part 20, and the fixing part 30.

The frame 10, the support part 20, and the fixing part 30 may be made of the same material resin. However, in the support structure 1 of the first embodiment, the frame 10 and the support part 20 may be made of a material different from and softer than a material of the fixing part 30, or only the frame 10 may be made of a material different from and softer than materials of the support part or the fixing part 30.

In this case, the frame 10, the support part 20, and the fixing part 30 are integrated by injection molding using different types of materials. Alternatively, the fixing part 30 may be separately made by injection molding from the frame 10 and the support part 20 and then bonded thereto.

Therefore, the frame 10 has lower rigidity than other parts, and the frame 10 is easily inserted by being folded or bent at the time of inserting the support structure of the first embodiment into the eyeball.

In addition, the fixing part 30 has a larger thickness or width than the frame 10 and the support part 20, such that an overall cross-sectional area increases, and rigidity of the frame and the support part may be reduced to be lower than rigidity of the fixing part 30.

The frame 10 has an entirely thin, flat cross-section and has a shape circularly extending in a plan view. The circular opening portion 11 is provided inside the frame 10, and the lens 110 of the artificial eye lens 100 is disposed in the opening portion 11.

The two fixing part 30 extend from the frame 10.

The fixing parts 30 are each placed on the same plane as the frame 10. The fixing part 30 extends straight in a tangential direction from the frame 10 and then is curved along a curved line in the same direction as the frame 10.

A first portion 31 of the fixing part 30, which adjoins the frame 10, extends straight and defines a portion disposed to extend toward the sclera in the eyeball when the first portion 31 is inserted into the eyeball.

A second portion 32 of the fixing part 30, which is spaced apart from the frame 10 and extends along a curved line, is formed in an arc shape entirely concentric with the frame 10, and a radius of curvature of the second portion 32 is equal to a radius of curvature of the sclera. When the support structure is implanted into the eyeball, a tip side of the second portion 32 is inserted into the sclera.

Meanwhile, all the second portions of the fixing parts of the support structure according to the first embodiment and a second embodiment to be described below each have a radius of curvature equal to the radius of curvature of the sclera so as to be easily inserted into the tunnels formed in the sclera. However, a radius of curvature of the second portion 32 may be smaller than the radius of curvature of the sclera so that the second portion 32 may come into close contact with the tunnel of the sclera while having a restoring force toward the inside of the sclera.

The two fixing parts 30 are disposed at an interval of 180 degrees while being opposite to each other with respect to the frame 10, such that the two fixing parts 30 are disposed linearly symmetrically with respect to a center point of the frame 10.

The four support parts 20 are disposed on the frame 10 at equal intervals of 90 degrees.

With reference to the cross-sectional view in FIG. 3, the support parts 20 each include: a first portion 21 extending from the frame 10 and inclined toward the outside of the plane on which the frame 10 is placed; and a second portion 22 extending from the first portion 21 and inclined toward the inside of the frame.

With the structure of the support part, a rim of one side surface 111 of the lens 110 of the artificial eye lens is supported on the first portion 21 of the support part, and a rim of a side surface 112 of the lens 110, which is opposite to one side surface 111 of the lens 110, is supported on the second portion 22 of the support part.

As described above, the support parts 20 restrain and support two opposite surfaces of the rim of the lens 110 of the artificial eye lens at four positions, such that the artificial eye lens 100 is kept coupled to the frame 10 by the support parts 20.

While a total of four support parts are provided in the support structure 1 of the first embodiment, only three support parts may be provided. In case that a width is large, two support parts may be provided and disposed to be symmetric with respect to each other.

The hole 34 is formed at the tip of the second portion 32 of the fixing part 30, one end of the thread 40 is coupled to the hole 34, and the needle 50 is coupled to the tip of the thread 40. The thread 40 and the needle 50 guide the second portion 32 of the fixing part 30 so that the second portion 32 is inserted into the sclera.

A process in which the artificial eye lens 100 is supported by the support structure 1 of the first embodiment will be described.

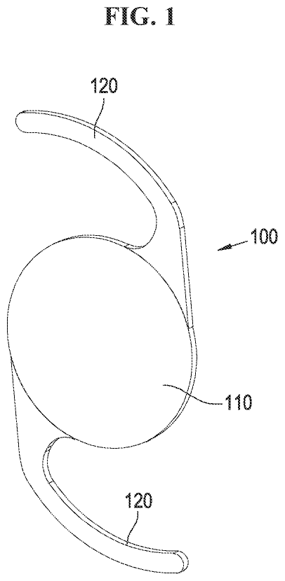

The artificial eye lens 100 is identical to that illustrated in FIG. 1 and has the lens 110, and two haptic parts 120 extending from a rim of the lens.

The lens 110 is placed to be concentric with the frame 10 and fixed to the support structure 1 by the frame 10 and the four support parts 20.

Rims of two opposite surfaces 111 and 112 of the lens are in contact with the first portion 21 and the second portion 22 of the support part 20. Because the support parts 20 restrain the lens 110 at the four positions, the lens 110 does not separate from the support structure 1 in any direction.

Meanwhile, the haptic parts 120 of the artificial eye lens protrude between the support parts 20 while departing from the frame 10.

The haptic parts 120 of the artificial eye lens 100 are provided to be supported in a capsular sac of a person's eyeball. Therefore, in case that the artificial eye lens 100 is supported in the person's eyeball by the support structure 1 of the first embodiment, the haptic parts 120 do not perform any function. However, the haptic parts 120 of the artificial eye lens do not hinder the support structure 1 of the first embodiment from being used for the artificial eye lens.

FIG. 4 illustrates a state in which the support structure 1 of the first embodiment is implanted into the eyeball, and the artificial eye lens 100 is supported on the support structure 1.

A state in which the artificial eye lens is supported by the support structure 1 of the first embodiment and implanted into the eyeball and a surgical procedure of making the above-mentioned state will be described with reference to FIG. 4.

To implant the artificial eye lens 100 into the eyeball, a cornea 201 is incised first, a tool is inserted into the incised portion, and a damaged eye lens and a damaged capsular sac are removed by suction.

Next, the support structure 1 according to the first embodiment of the present invention is inserted through the incised portion of the cornea 201.

The frame 10 is inserted by being folded or bent at the time of inserting the support structure 1 through the incised portion of the cornea 201. After the frame 10 is disposed rearward of an iris 202, the frame 10 is unfolded and disposed to be concentric with a pupil 204.

The support structure of the first embodiment is made of a material with high elasticity, and particularly, the frame 10 is softer or has lower rigidity than the fixing part 30, such that the frame 10 is easily folded and inserted into the incised portion of the cornea 201. Because the fixing part 30 is relatively hard or has high rigidity, the fixing part 30 is advantageous in supporting the support structure 1 in the eyeball.

Two holes (not illustrated) are formed in a sclera 203 so that the second portions 32 of the fixing parts 30 may penetrate the two holes and protrude to the outside. The needles 50 are extracted to the outside through the holes.

The frame 10 and the support parts 20 of the support structure 1 are disposed rearward of the iris 202. When the needles 50 exposed to the outside of the sclera 203 are pulled, the threads 40 coupled to the needles are pulled, such that the tips of the second portions 32 of the fixing parts are exposed to the outside of the sclera.

The tunnels (not illustrated), into which the tips of the second portions 32 of the fixing parts are inserted, are formed in advance in the sclera 203, and the tips of the fixing part 30 are inserted into the tunnels.

As necessary, a groove, in which the second portion 32 of the fixing part is placed, may be formed by incising an outer surface of the sclera between the tunnel and the hole of the sclera 203 penetrated by the fixing part. The groove may be sutured after the tip 32 of the fixing part is inserted into the tunnel.

After the tip of the fixing part 30 is exposed to the outside of the sclera 203, the thread 40 and the needle 50 are removed from the fixing part 30.

With the above-mentioned configuration and operation, the fixing part 30, which is very thin and has elasticity, may be disposed while penetrating the sclera 203, and the tip may be easily inserted into the tunnel during the surgical procedure in which the artificial eye lens and the support structure 1 of the first embodiment are inserted into the eyeball.

After the artificial eye lens 100 is inserted through the incised portion of the cornea 201, the rim of the lens 110 is fitted with the support part 20, such that the artificial eye lens 100 is fixed to the support structure 1 of the first embodiment.

As described above, as illustrated in FIG. 4, the frame 10 and the support part 20 of the support structure 1 are disposed rearward of the iris 102, the fixing part 30 extends from the frame 10, the second portion 32 penetrates the sclera 203, the tip of the fixing part 30 is inserted and fixed into the tunnel, and the lens 110 of the artificial eye lens, which is disposed to be concentric with the frame 10 of the support structure and supported by the support structure 1, is implanted into the eyeball.

Meanwhile, in the above-mentioned description, the configuration has been described in which the artificial eye lens 100 is supported by the support structure 1 by the process of inserting the artificial eye lens 100 after fixing the support structure 1 to the eyeball. However, another situation may occur in an actual surgical procedure.

For example, during a surgical procedure of suctioning and removing the eye lens of the eyeball and inserting the artificial eye lens into the capsular sac so that the haptic part of the artificial eye lens is supported in the capsular sac, a situation may occur in which the capsular sac is damaged, and the artificial eye lens already inserted into the capsular sac and exiting in the eyeball cannot be supported. In addition, the artificial eye lens implanted into the eyeball sometimes deviates into a vitreous cavity (106 in FIG. 4) because of an injury, a disease, or the like without being maintained in position.

In this case, the support structure 1 of the first embodiment is inserted through the incised portion of the cornea 201, and the above-mentioned process is performed.

Meanwhile, the support part 20 of the support structure 1 may be disposed to be directed toward the rear side, i.e., the side opposite to the cornea 201. That is, in the state illustrated in FIG. 3, the support part 20 is disposed to be directed toward the front side, i.e., the cornea 201, and the frame 10 is disposed at the rear side. However, on the contrary, the frame 10 is disposed at the front side, and the support part 20 is disposed to be directed toward the rear side, i.e., the retina 205.

As described above, the artificial eye lens 100, which deviates into the vitreous cavity 206, may be easily fitted with the support part 20 of the support structure 1 disposed rearward of the iris 203.

However, even in case that the support structure 1 of the first embodiment is disposed in a state illustrated in FIG. 3, the artificial eye lens 100, which deviates into the vitreous cavity 206 may be moved to the support part 20 of the support structure through the opening portion 11 of the frame 10 and then fixed.

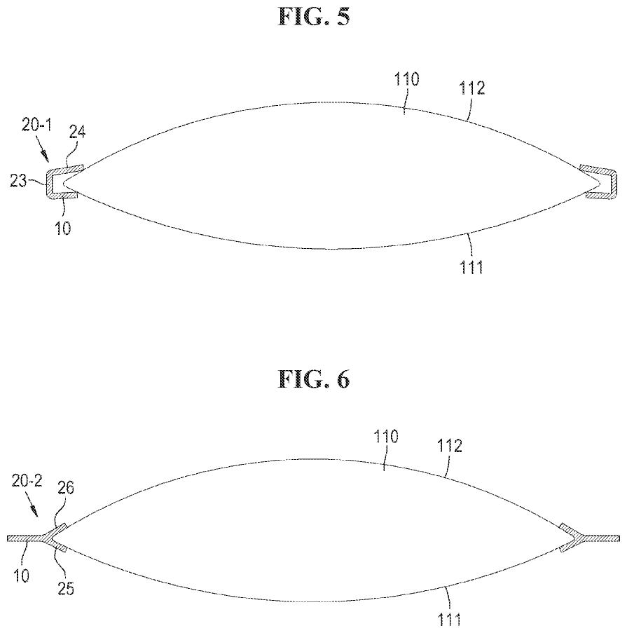

Modified examples of the first embodiment will be described with reference to FIGS. 5 and 6.

The support structures of the modified examples illustrated in FIGS. 5 and 6 are identical to the support structure of the first embodiment, except for structures of support parts 20-1 and 20-2.

In the support structure of the first modified example illustrated in FIG. 5, a support part 20-1 includes a first portion 23 extending perpendicularly from a surface of the frame 10, and a second portion 24 extending from the first portion toward the inside of the frame 10.

With the above-mentioned structure, the rim of one side surface 111 of the lens 110 of the artificial eye lens is supported on the surface of the frame 10, and the rim of the other side surface 112 is supported by the second portion 24 of the support part 20′.

In the support structure of the second modified example illustrated in FIG. 6, a first portion 25 and a second portion 26 of the support part 20-2 extend to be inclined in opposite directions from an end, i.e., an inner periphery of the opening portion 11 of the frame 10.

Two opposite surfaces of the rim of the lens 110 of the artificial eye lens are respectively in contact with the first portion 25 and the second portion 26, such that the lens 110 is restrained and supported by the support part 20-2.

Even in the modified examples, like the first embodiment illustrated in FIGS. 2 to 3, the lens 110 of the artificial eye lens is restrained and supported by the support structure by means of the support parts 20-1 and 20-2 and the frame 10, the support parts 20-1 and 20-2 may also be identical in arrangement and number to those in the first embodiment.

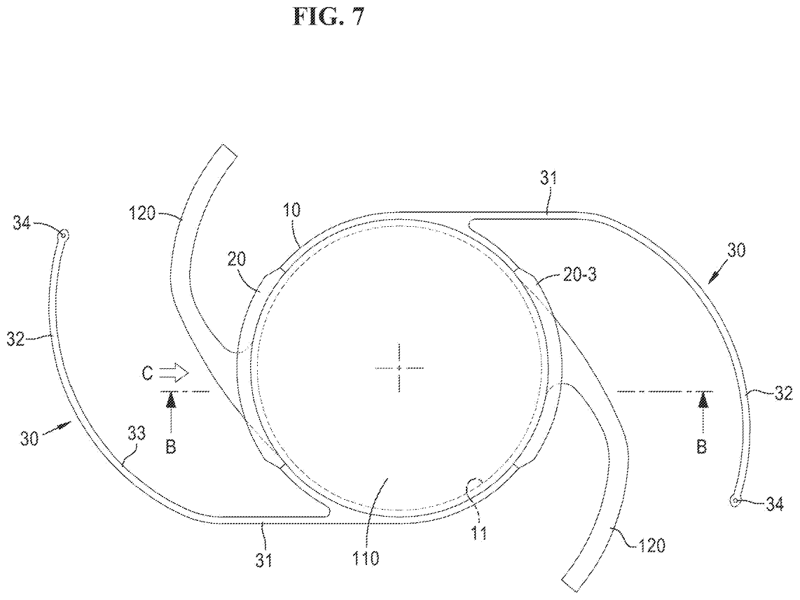

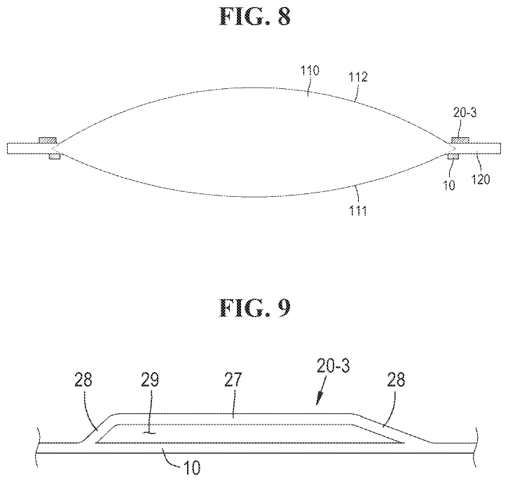

Next, a support structure 2 of a second embodiment will be described with reference to FIGS. 7 to 9.

The support structure 2 of the second embodiment is also identical to the first embodiment in that the support structure 2 includes the frame 10, support parts 20-3, and the fixing parts 30. However, the second embodiment is different from the first embodiment in that a thread and a needle are not coupled to the tip of the fixing part 30, and the second embodiment is different in a configuration of the support part 20-3 from the first embodiment.

However, even in the support structure of the second embodiment, a thread and a needle may be coupled to ensure convenience for a surgical procedure.

A configuration of the support part 20-3 of the support structure 2 of the second embodiment will be described.

Two support parts 20-3 are provided in the support structure 2 of the second embodiment.

FIG. 9 is a view when viewed from arrow C in FIG. 7 and illustrates only the support structure in which the artificial eye lens 100 is excluded. With reference to FIG. 9, the support part 20-3 includes: two second portions 28 extending and inclined from the surface of the frame 10; and a first portion 27 disposed between the second portions 28, extending in parallel with the surface of the frame 10, and spaced apart from the surface of the frame 10 at a predetermined distance.

Therefore, the first and second portions 27 and 28 of the support part 20-3 define a ring 29 together with the surface of the frame 10. The haptic part 120 of the artificial eye lens 100 is inserted into the ring 29 and protrudes to the outside of the support structure 2.

One side surface of the lens 110 of the artificial eye lens 100 is placed on the surface of the frame 10. The two haptic parts 120 protrude from the frame 10 of the support structure 2 while passing through the rings 29 defined by the support parts 20-3 and the surface of the frame 10.

Therefore, the artificial eye lens 100 is restrained and supported by the support structure 2.

A process of implanting the support structure 2 of the second embodiment into the eyeball and supporting the artificial eye lens 100 on the support structure is substantially identical to that in the first embodiment.

However, the second embodiment is different from the first embodiment in that the two haptic parts 120 of the artificial eye lens 100 need to be inserted into the rings 29 defined by the support parts 20-3 of the support structure after the support structure 2 of the second embodiment is implanted into the eyeball.

In addition to the artificial eye lens having the two haptic parts, there is an artificial eye lens having four haptic parts. In the case of the artificial eye lens, only the two haptic parts are inserted into the rings 29 of the support parts 20-3, and the remaining haptic parts protrude to the outside of the frame 10.

Meanwhile, in the second embodiment, the ring 29 through which the haptic part 120 of the artificial eye lens passes and is supported is defined by the frame 10 and the support part 20-3. However, the support part may have a ring shape, and the support part may be coupled to the frame. In addition, the shape of the ring is not limited. However, the ring may be provided over a large width of the frame 10 so that the haptic part may be easily inserted and supported regardless of the arrangement and shape of the haptic part 120.

While the configurations and the operations of the exemplary embodiments of the present invention have been described above, the present invention is not limited to the exemplary embodiments, various modifications and addition of constituent elements are enabled within the scope defined by the appended claims, and the modified support structure and the support structure, to which the constituent elements are added, fall within the scope of the present invention.

1: A support structure for supporting an artificial eye lens in an eyeball, the artificial eye lens being implanted into the eyeball so as to be substituted for an eye lens, the support structure comprising:
a frame extending in an arc shape concentric with a lens of the artificial eye lens, having an opening portion therein, disposed adjacent to a rim of the lens of the artificial eye lens or disposed to support a rim of one side surface of the lens;
a plurality of support parts disposed to be spaced apart from one another in a circumferential direction of the frame and configured to support the artificial eye lens to prevent the artificial eye lens from separating from the support structure; and
a plurality of fixing parts extending outward from the frame in a diameter direction of the frame and having tips inserted and fixed into tunnels formed in a sclera of the eyeball,
wherein the artificial eye lens comprises the lens, and a plurality of haptic parts extending outward from the lens in a diameter direction of the lens and configured to come into contact with a capsular sac, and
wherein the artificial eye lens is disposed on the frame so that the haptic parts each protrude between the support parts.

2: The support structure of claim 1, wherein the support parts each comprise:
a first portion extending from the frame and being in contact with one side surface of the lens of the artificial eye lens that faces one surface of the frame; and
a second portion extending from a tip of the first portion and being in contact with a side surface of the lens opposite to one side surface of the lens, and
wherein the artificial eye lens is supported as rims of two opposite surfaces of the lens of the artificial eye lens are in contact with the first and second portions of the support part.

3: The support structure of claim 1, wherein the support parts each comprise:
a first portion extending from the frame and being in contact with one side surface of the lens of the artificial eye lens that faces one surface of the frame; and
a second portion extending from the frame and being in contact with a side surface of the lens opposite to one side surface of the lens, and
wherein the artificial eye lens is supported as rims of two opposite surfaces of the lens of the artificial eye lens are in contact with the first and second portions of the support part.

4: The support structure of claim 1, wherein a circumference of one side surface of the artificial eye lens is in contact with one surface of the frame, and each of the support parts extends from the frame and is configured to come into contact with a side surface of the artificial eye lens opposite to one side surface of the artificial eye lens, such that the artificial eye lens is supported as rims of two opposite surfaces of the lens are in contact with one surface of the frame and the support parts.

5: A support structure for supporting an artificial eye lens in an eyeball, the artificial eye lens being implanted into the eyeball so as to be substituted for an eye lens, the support structure comprising:
a frame extending in an arc shape concentric with a lens of the artificial eye lens, having an opening portion therein, disposed adjacent to a rim of the lens of the artificial eye lens or disposed to support a rim of one side surface of the lens;
a plurality of support parts disposed to be spaced apart from one another in a circumferential direction of the frame and configured to support the artificial eye lens to prevent the artificial eye lens from separating from the support structure; and
a plurality of fixing parts extending outward from the frame in a diameter direction of the frame and having tips inserted and fixed into tunnels formed in a sclera of the eyeball,
wherein the artificial eye lens comprises the lens, and a plurality of haptic parts extending outward from the lens in a diameter direction of the lens and configured to come into contact with a capsular sac,
wherein the support parts are each formed in a ring shape fixed to the frame, and
wherein the artificial eye lens is supported as the haptic parts of the artificial eye lens are inserted into the rings.

6: The support structure of claim 1, wherein the entire support structure is formed by injection molding using synthetic resin, and
wherein the fixing part has higher rigidity than the frame.

7: The support structure of claim 1, wherein a tip side of the fixing part has the same curvature as a sclera and is formed along a curved line concentric with the frame.

8: The support structure of claim 1, wherein a thread and a needle are sequentially coupled to the tip of the fixing part.
